(19) United States
(12) Patent Application Publication
(10) Pub. No.: US 2004/0132650 A1
(43) Pub. Date: Jul. 8, 2004

(54) Human growth hormone for treating children with abnormal short stature and kits and methods for diagnosing gs protein dysfunctions

(76) Inventor: Francis De Zegher, Tervuren (BE)

Correspondence Address: CLARK & ELBING LLP, 101 FEDERAL STREET, BOSTON, MA, 02110, US

(21) Appl. No.: 10/469,783

(22) PCT Filed: Mar. 18, 2002
(86) PCT No.: PCT/BE02/00038

(30) Foreign Application Priority Data
Mar. 16, 2001 (GB) 0106565.5

(51) Int. Cl. A61K 38/27

(52) U.S. Cl. 514/12

(57) ABSTRACT

The present invention presents the use of human Growth Hormone for the manufacture of a medicament for the treatment of abnormal short stature, said short stature being characterized by a Gs protein pathway dysfunction. The invention further present diagnostic methods and diagnostic kits for the determination of Gs pathway dysfunction.

The present invention relates to a novel use of human Growth Hormone and functional variants thereof, and more specifically to the use for the manufacture of a medicament for treating a condition of abnormal short stature in humans. In particular the present invention relates to test methods and diagnostic kits for detecting Gs protein pathway dysfunction in humans, the latter being identified as responsible and causative for certain conditions of abnormal short stature. These test methods may be functional assays involving agonists or antagonists of Gs protein-coupled receptors, or genetic assays involving detecting alterations in Gs proteins or in genes encoding the said Gs proteins or components of the said genes. This invention is therefore useful in increasing the growth rate of children wherein the condition of abnormal short stature is identified as being linked to Gs protein dysfunction.

### BACKGROUND OF THE INVENTION

Short stature in humans may be caused by several disorders such as Growth Hormone deficiency (hereinafter referred as GHD). In this group of persons, the deficiency may have known causes for a number of individuals. However the underlying cause of GHD is often not clearly understood.

Short stature in non-GHD individuals may occur in persons who were born with a normal weight and length at birth. For these persons also, an underlying cause for short stature may be known, such as multiplet pregnancy. Nevertheless, a significant number of persons with short stature are still classified as iodiopathics, meaning that the cause of short stature is unknown.

Therefore, to date, there is no proper currently available diagnosis for the cause of short stature in a significant number of persons with short stature and consequently there is no rational medical treatment in order to accelerate their growth or to normalize their stature.

Growth hormone (hereinafter referred as GH) is currently administered in childhood to treat short stature related to GHD. Turner syndrome or renal failure. Most children with one of these conditions are of normal size in early life but present gradual growth failure resulting in short stature by late childhood, and, if left untreated, reach an adult height significantly below both target and normal range. Accordingly GH therapy is now mostly initiated in the late childhood of children with GHD, the prime therapeutic objective of such treatment being to optimize the late stage of growth and thus safely normalizing adult height.

Proportionate short stature, accompanied by a decreased growth velocity, is the most important clinical finding to support the diagnosis of growth hormone deficiency (GHD). Additional findings of delayed bone maturation and the absence of bone dysplasias and chronic diseases are additional criteria. Adequate function of the GH pathway is needed throughout childhood to maintain normal growth. While most newborns with GHD have normal length and weight, those with complete absence of GH due to GH gene deletions may have a birth length shorter than expected for their birth weight. The low linear growth of infants with congenital GHD becomes progressively retarded with age and some may have micropenis or fasting hypoglycemia. In those with isolated GHD (hereinafter referred as IGHD), skeletal maturation is usually delayed in proportion to their height retardation.

Apart from mutations in the gene coding for GH, an insufficient level of GH may also be caused by a dysfunction of proteins such as Growth Hormone-Releasing hormone (hereinafter referred as GHRH) or its receptor. Other disorders may be responsible for children with short stature. In particular, there are also known children with short stature but wherein normal serum peak levels of Growth Hormone (i.e. above 10 ng/ml) are measured.

Albright hereditary osteodistrophy (AHO)-specific dysmorphologies are often associated with resistance to parathyroid hormone and other hormones (in which case the disorder is often named as pseudohypoparathyroidism type 1a (hereinafter PHP). When AHO occurs without hormone resistance in families with pseudohypoparathyroidism type 1a, the disorder is described as pseudopseudohypoparathyroidism (hereinafter PPHP). PPHP and PHP appear to be less severe variants of AHO, the latter exhibiting hyper parathyroidism with ectopic ossifications and skeketal defects, optionally together with decreased intelligence.

At least three disorders with features of dysregulated osteogenesis are associated with somatic or germ-line mutations in GNAS1, the gene that encodes Gsa, the alpha sub-unit of Gs. The McCune-Albright (MCA) syndrome is caused by post-zygotic mutations that activate Gsa and cause constitutive (hormone-independent) activation of adenylyl cyclase, whereas AHO and plate-like osteoma cutis are caused by inactivating germ-line GNAS1 mutations. It was recently shown that another disorder of osteogenesis, progressive osseous heteroplasia (hereinafter POH) is due to inactivating mutations in the same gene, according to Shore et al. in N. Eng. J. Med. (2002) 346:99-106.

The GNAS1 gene encodes the alpha subunit of the guanine nucleotide-binding protein Gs, which couples signaling through peptide hormone receptors to cAMP generation. GNAS1 mutations underlie the hormone resistance syndrome pseudohypoparathyroidism type Ia (PHP-Ia), so the maternal inheritance displayed by PHP-Ia has raised suspicions that GNAS1 is imprinted. Despite this suggestion, in most tissues Gsalpha is biallelically encoded. In contrast, the large G protein XLalphas, also encoded by GNAS1, is paternally derived. Two upstream promoters, each associated with a large coding exon, lie only 11 kb apart, yet show opposite patterns of allele-specific methylation and monoallelic transcription. The more 5′ of these exons encodes the neuroendocrine secretory protein NESP55, which is expressed exclusively from the maternal allele. The NESP55 exon is 11 kb 5′ to the paternally expressed XLalphas exon. The transcripts from these two promoters both splice onto GNAS1 exon 2, yet share no coding sequences. Despite their structural unrelatedness, the encoded proteins, of opposite allelic origin, both have been implicated in regulated secretion in neuroendocrine tissues. Remarkably, maternally (NESP55), paternally (XLalphas), and biallelically (Gsalpha) derived proteins all are produced by different pattems of promoter use and alternative splicing of GNAS1, a gene showing simultaneous imprinting in both the paternal and maternal directions.

Due to some of the characteristics (such as cost and duration) of a Growth Hormone treatment, populations of short stature children with one of the aforesaid disorders cannot benefit from the same treatment as the populations of children with GHD or IGHD in the absence of a clear and predictable rationale of the reasons for which such a treatment could be useful. Therefore, there is still a strong need in the art for a reliable diagnostic method contributing to select those children with short stature, in particular those without GHD or IGHD, who could consistently benefit from a treatment with human Growth Hormone in order to safely normalize adult height or to minimize the height deficit when compared with normal range.

### SUMMARY OF INVENTION

A first object of the present invention is the treatment of abnormally short stature in children identified by means of functional or genetic assays.

As a part of the invention the surprising and unprecedented correlation is made in a number of persons with short stature between the occurrence of short stature and the occurrence of a dysfunction of the Gs protein signaling pathway as ascertained by means of a functional or a genetic assay for Gs protein signaling.

It is another part of the invention to use the main features of a Gs protein functional assay originally linked to bleeding disorders or genetic disorders such as AHO, for diagnosing the cause of unexplained short stature, a condition that is unrelated to bleeding disorders or said genetic disorder.

It is another part of the invention that within persons with short stature characteristics, mutations are encountered within the coding region of the GNAS1 gene encoding for the Gs alpha subunit and the Gs XL alpha subunit, components of the Gs protein pathway.

The GNAS1 coding sequence and its regulatory elements are the subject of extensive study related to genetic disease such as MCA, POH, AHO, PPH, PPHP. These genetic diseases exhibit complex clinical phenotypes of which reduced growth is only a part. By definition, these genetic diseases have not been correlated with the phenotype of just short stature.

It is therefore an aspect of the invention to assign a fraction of those persons with short stature, which were previously classified as idiopathic, as persons having a dysfunction in the Gs protein pathway.

It is further a part of the invention to recognize short stature as a previously unrecognized mild variant of a more readily evident phenotype fitting into the definitions of the more severe disorders mentioned above that include short stature as one of their consistent features

The present invention is based on the unexpected finding that the Gs protein pathway dysfunction is the short stature causative deficiency common to a significant number of persons being currently defined as having idiopathic short stature, and that this deficiency can be determined by means of functional or genetic assays. As a result of this first finding, the invention is based on the experimental evidence that children identified according to this assay can be successfully treated with human Growth Hormone in order to safely normalize height or at least approximate the normal range of height in childhood or adulthood. Said children can be male as well as female and includes individuals during puberty.

Thus the invention discloses variants of Gs alpha protein pathway dysfunction as a cause of abnormally short stature in humans, and shows that the administration of an effective amount of human growth hormone is a safe treatment to predictably induce normalization of stature in these children.

Furthermore, this invention provides the treatment of humans with Growth Hormone (e.g. somatotropin) for human growth failure or short stature associated with a disorder of transmembrane signaling through G protein coupled receptors, including disorders of G protein pathway function evidenced by functional testing (e.g. in erythrocytes or thrombocytes) and/or those identified by genetic defects such as epigenetic mutations or polymorphisms in the GNAS1 gene on chromosome 20q13 or within its alternative promoters, in particular Xlalphas or NESP55.

The present invention will now be described with reference to the following drawings.

### BRIEF DESCRIPTION OF THE DRAWINGS

FIG. 1 shows observations of the height growth (in cm) of a girl treated with human growth hormone according to the invention from the age of 47 months.

FIG. 2 shows observations of the height growth (in cm/year) of a girl treated with human growth hormone according to the invention from the age of 47 months.

FIG. 3 shows observations of the height growth (in cm) of a boy treated with human growth hormone according to the invention from the age of 10,3 years.

FIG. 4 shows observations of the height growth (in cm/year) of a boy is treated with human growth hormone according to the invention from the age of 10,3 years.

### DEFINITIONS

The term “G proteins” as used herein means receptors which are associated with the inner surface of the plasma membrane and couple receptors to their respective effectors, which regulate the metabolism of second messengers, e.g. cAMP. G proteins consist of three polypeptide sub-units named alpha, beta and gamma respectively. The alpha sub-unit binds guanine nucleotides with high affinity and specificity. The beta- and gamma polypeptides are associated to form a dimer. All GPCRs act by promoting the release of tightly bound GDP from the alpha sub-units, enabling GTP to bind and activate the G protein (GTPase cycle) resulting in dissociation of the alpha sub-unit from the beta-gamma dimer and, by stimulating the effector, in generation of second messengers according to Spiegel in Horm. Res. (1997) 47:89-96. So far, 16 mammalian alpha sub-unit genes have been identified, which can be divided into four subfamilies: Gₛ, G_{q}, Gᵢ, G₁₂. In general, Gₛ and G_{q} stimulate the associated effector, whereas Gᵢmediates inhibition. A large number of receptors stimulate (Gₛ) or inhibit (Gᵢ) adenylyl-cyclase, resulting in increase or decrease of cAMP inside the cell. Gₛalpha is encoded by the gene GNAS1 which is located on chromosome 20q13.1-13.2. It contains at least 13 coding exons and 20 introns, and spans over 20 kilobase pairs. Recently, it has been shown that this gene is imprinted in a promoter-specific fashion. In humans, GNAS1 has 2 different promoters (Xlalphas and NESP55) and first exons which can splice alternatively to exon 2 of GNAS1. These promoters display opposite patterns of allele specific methylation.

The term “Growth Hormone” as used herein means any variant that has a normal human Growth Hormone (hGH) activity, although mature hGH (a single polypeptide chain of 191 amino acids having a molecular weight of 22,124) is more preferable in terms of, for example, antigenicity. For example, native preparation purified from the pituitary gland, Met-hGH in which a methionine residue is attached to the N-terminus of native hGH, or even recombinant hGH variants may be used as long as they have hGH activity. Genbank sequence accession P01241 shows the human precursor sequence of 217 amino-acids and also shows from amino-acids 27 to 217 the mature active chain also known as somatotropin. Therefore, the term “human Growth Hormone (hGH)” as used herein encompasses all hGH derivatives having hGH activity regardless of their origins or production processes.

SGA is an abbreviation for “small for gestational age” and refers to individuals with a standard deviation score equal or below −2 for weight and/or length at birth.

“Abnormal short stature” is defined herein as the stature of individuals with a standard deviation score equal or below −2 for weight and/or length at the time period when the functional or genetic screening assay of this invention is performed. (according to Karlberg et al. (1976) in Acta Paediatr. Scand. Suppl. 258, 7-76).

“Gene” as used herein relates to a segment of DNA involved in producing a polypeptide chain and comprises coding sequences (exons), intervening sequences (introns) and 5′ and 3′ regulatory sequences. Within this invention “GNAS1 gene” relates to the different patterns of coding, intervening and regulatory elements for the production of the proteins Gs alpha, XL alpha and NESP55 and naturally occurring splice variants of said proteins. “Functional variant” as used herein refers to protein having a different amino-acid sequence compared to the sequence of the wild-type protein but having a similar specific activity.

“Relevant control population” as used herein means a population of human individuals of the same age and sex and taking in account ethnic and/or national background. The national and/or ethnic background is reflected in growth charts as provided for example in scientific publications or by national health organizations or national institutes of statistics such as for instance the American National Center of Health Statistics (NCHS) of the Centre of Disease Control (CDC).

“Functional assay” refers to an assay where a dysfunction of a protein in a pathway is evaluated by the identification of a change in a downstream is effect of the said pathway, such as the production of a metabolite or a measurable morphological modification related to the pathway. It should be understood that, while the treatment and the medicament of the invention only apply to persons wherein the epiphysial growth plates are not fused, the diagnosis of a Gs protein pathway dysfunction may be performed on any human being, regardless of his age.

“Modifying protein” as used in this invention relates to enzymes which can methylate or de-methylate DNA, and which are known as respectively DNA methyltransferases and DNA methylesterases. Protein modification at the molecular level deals with any of the following: mutations, insertions, deletions and inversions.

“Proteins encoded by the GNAS1 gene” as used herein relates to all naturally encoding splice variants encoded by the GNAS1 gene.

“Protein of the Gs pathway” as used in this invention relates to any protein in Gs receptor signaling which is functionally located downstream of the said receptor.

“Gs protein’ relates to the trimeric protein complex comprising a Gs alpha sub-unit or a splice variant which complexes with a beta-gamma G protein dimer.

“Growth Hormone deficient” or “Growth Hormone deficiency” relates to a condition diagnosed on the basis of a composite process requiring comprehensive clinical and auxological assesment, combined with biochemical tests of the GH-lnsulin like growth factor (IGF) axis and radiological evaluation as extensively documented by the GH Research Society in J. Clin. Endocrinol.(2000) 85:3990-3993. Traditionally, a peak GH concentration below 10 μg/L has been used to support the diagnosis of Growth Hormone deficiency.

“Allele” is one of several alternative forms of a gene occupying a locus on a chromosome.

“Locus” is the position on the chromosome at which the gene for a particular feature resides. The locus may be occupied by any one of the alleles of the gene.

“Complex locus” refers to a locus, the genetic properties of which are inconsistent with the function of a gene representing a single protein Complex loci are usually more than 100 kb long.

“Gs alpha protein” as used herein refers to a protein encoded by the GNAS1 gene coding for the human guanine nucleotide-binding protein Gs alpha sub-unit with a length of 394 amino-acids with Genbank Accession number NP_{—}000507 but also to naturally occurring splice variants spliced thereof. An example of such a splice variant is the protein encoded by the GNAS1 gene, wherein amino-acids 73 to 86 are absent and wherein the amino-acids Glu and Gly at positions 71 and 72 are replaced respectively by Asp and Ser. Another example of such a splice variant is the GsXI-alpha-s protein with a length of 441 amino-acids with Genbank Accession number CAA12165.

“mutations or functional polymorphisms” refers to one or more modifications at the DNA level such as substitutions, insertions, deletions, inversions, regardless of their size.

### DETAILED DESCRIPTION OF THE INVENTION

In a first aspect, the present invention provides the use of human Growth Hormone, or a functional variant thereof, for the manufacture of a medicament for the treatment of a child with a condition of abnormal short stature in order to increase the growth rate of the said child, wherein the said condition is characterized by a Gs protein pathway dysfunction identifiable by a functional or genetic assay.

According to the invention, the said condition of abnormal short stature may be further characterized by human Growth Hormone deficiency. However, the said condition of abnormal short stature preferably does not further include human Growth Hormone deficiency.

According to one specific embodiment of the invention, the said condition of abnormal short stature may be characterized by a body weight and/or length at birth which is more than two standard deviations below average of the relevant control human population. However, this requirement may also be met at any time after birth as long as epiphyseal growth plates are not fused.

According to the invention, the said Gs protein pathway dysfunction may be a Gs alpha protein dysfunction or a Gs beta protein dysfunction. The stimulation of Gs protein pathway leads to the stimulation of adenylate cyclase, the enzyme which converts ATP into cyclic AMP (cAMP). Therefor the measurement of labeled cAMP out of labeled ATP is well known assay for correlating Gs protein functioning with cAMP production. More reliable assays are performed on cells, permeabilized cells or cell membranes. For this purpose any source of cells can be used. For example, Gs protein pathway functioning can be determined by measuring the Gs activity of erythrocyte membranes as described by Levine et al. (1986) J. Clin. Endocrinol. Metab. 62, 497-502 or by measuring adenylate cyclase activities and cAMP accumulation in fibroblasts propagated for skin biopsies as described in Bourne et al (1981) J. Clin. Endocrinol. Metab 53, 636-640. In addition it is well known that prostacyclins stimulate Gs protein signaling in platelets leading to an increased cAMP production and to a reduction in platelet aggregation (see Cardiovascular thrombosis 2^{nd} Ed (1998) pages 32-34 Eds Verstraete, Fuster and Topol. Lippincott Raven Publishers, Philadelphia). Based on the above mentioned behavior of platelets towards Gs pathway agonists and antagonist assays can be carried out in order to measure platelet aggregation and/or CAMP production. As an example of this type of assay reference is made to Freson et al. (2001) in Thromb. Haemost. 86, 733-738.

According to one specific embodiment of the invention, the functional assay which may be used in identifying the Gs protein pathway dysfunction comprises a thrombocyte aggregation-inhibition test indicating a change in G protein mediated signaling by at least one inducer, preferably a thrombocyte aggregation-inhibition test indicating a loss in G protein mediated signaling by at least two inducers; More preferably, the said inducers are selected from the group consisting of prostaglandin E1, iloprost and adenosine. According to an alternative embodiment of the invention, the functional assay which may be used in identifying the Gs protein pathway dysfunction comprises the steps of:

(a) providing a tissue or cell source isolated from the said child with a condition of abnormal short stature,

(b) contacting the said tissue or cells obtained from the said cell source with one or more agonists or antagonists of Gs protein-coupled receptors,

(c) determining the response to all said agonists or antagonists of Gs protein-coupled receptors in the said cells or tissue,

(d) comparing the response obtained in step (c) with a reference response to all said agonists or antagonists of Gs protein-coupled receptors in the cells or tissue of a control human population, and

(e) determining whether the response obtained in step (c) is significantly different from the said reference response.

Within this embodiment, preferably the said agonists or antagonists of Gs protein-coupled receptors are platelet-aggregation antagonists which may for instance be selected from the group consisting of prostaglandin E1, adenosine and chemically stable prostacycline analogues (such as for example iloprost, cicaprost, ataprost, beraprost, ciprostene, taprostene, naxaprostene and the like).

According to one specific embodiment of the invention, the genetic assay which may be used in identifying the Gs protein pathway dysfunction consists in detecting one or more alterations in one or more Gs proteins or in one or more genes encoding the said Gs proteins or components of the said genes.

For instance, when the Gs protein pathway dysfunction is a Gs alpha protein dysfunction, the said genetic assay preferably consists in detecting one or more alterations related to the Gs alpha protein or the GsXL alpha protein, the said alterations being selected from the group consisting of:

mutations or functional polymorphisms in the human GNAS1 coding sequence and/or human GNAS1 regulatory sequence. These can be identified by sequencing genomic or cDNA, or can be detected by other currently available techniques such as single-stranded conformation polymorphism assay (SSCA), clamped denaturing gel electrophoresis (CDGE) heteroduplex analysis (HA), chemical mismatch cleavage (CMC) protein truncation assay, allele specific oligonucleotide (ASO) hybridization can be utilized.

variations in the methylation pattern of the human GNAS1 locus. These can be detected via methylation specific restriction enzymes. These enzymes are not able to cleave DNA at their recognition site when it is methylated. Southern blotting of DNA treated with methylation sensitive and methylation insensitive restriction enzymes are used to study and compare methylation assays. Reference is made to Hayward et al (1998) in Proc. Natl. Acad. Sci USA 95, 15475-15480, wherein the methylation patterm of the GNAS locus is determined.

variations in the expression level of a human GNAS1 encoded protein. The levels of GNAS encoded proteins can be detected directly at the protein level with monoclonal or polyclonal antibodies against GNAS encoded proteins in a quantitative protein detection analysis such as ELISA. Protein levels can also be determined indirectly via Reverse Transcriptase PCR (RT-PCR) using specific primers for a mRNA molecule of a GNAS encoded protein of choice.

different splicings of the human GNAS1 transcript. Splicing events can be detected at several levels, such as verification of the observed Mr of a protein with a predicted Mr of a protein obtained by a certain splice variant. Alternatively, the presence and the size a mRNA can be detected by Northern blot analysis or by RT PCR.

mutations or functional polymorphisms in an alternative human GNAS1 promoter, these are detected by sequencing the genomic sequence of the promoter region.

and mutations in a modifying protein of the human GNAS1 locus. These can be detected by sequencing the genes encoding DNA methyltransferases and DNA methylesterases.

More specifically, the GNAS1 coding sequence mutation may be a mutation within the triplet coding for Arg231 of Gs alpha protein, e.g. the GNAS1 coding sequence mutation may result in a Arg231Cys mutation in the Gs alpha protein. The GNAS1 coding sequence mutation may also be an insertion into the internal repeat region of the GsXL protein encoded by the human GNAS1 gene and/or one or more substitutions within said internal repeat region. Alternatively, the GNAS1 coding sequence mutation may be a 36 bp insertion leading to a duplication of repeat 7 or repeat 8 structure of the internal repeat region of the GsXL protein encoded by the human GNAS1 gene and/or a substitution of Ala138 with Asp and/or a substitution of Pro161 with Arg in the GsXL protein.

When the Gs protein pathway dysfunction is a Gs beta protein dysfunction, then the genetic assay which may be used in identifying the Gs protein pathway dysfunction consists in detecting one or more alterations in the Gs beta protein or a gene encoding said protein.

In order to satisfactorily increase the growth rate of the child with abnormal short stature, the human Growth Hormone, or a functional variant thereof, should preferably be used in the said treatment in an amount between 36 and 64 μg per kg bodyweight per day. Surprisingly, this amount is significantly higher than the 25 to 35 μg per kg bodyweight per day recommended when human Growth Hormone is administered for the treatment of growth retardation or other conditions of Growth Hormone deficiency.

Preferably, children with a condition of abnormal short stature concerned by the present invention should preferably be aged at least 2 years and until a condition where epiphyseal growth plates are not fused. Obviously, in order to gain optimal benefit from the medicament and treatment of this invention, treatment should preferably be started as soon as the condition of a body weight and/or length being more than two standard deviations below average of the relevant control human population is detected.

Another aspect of this invention relates to a test method for use in diagnosing a disease or abnormal condition related to a Gs protein pathway dysfunction in a human being, the test being carried out on a tissue sample or a cell source from a human being, wherein the test comprises detecting one or more alterations in one or more Gs proteins or in one or more genes encoding the said Gs proteins or components of the said genes. The invention also provides a diagnostic method for diagnosing a Gs protein pathway dysfunction or a disease or abnormal condition related thereto in a human being, comprising the step of performing onto the said human being, or a tissue sample or a cell source thereof, at least a genetic test consisting in detecting one or more alterations in one or more Gs proteins or in one or more genes encoding the said Gs proteins or components of the said genes. Preferably, the said abnormal condition is defined as an abnormal short stature characterized by a body weight and/or length which is more than two standard deviations below average of the relevant control human population.

According to this test or diagnostic method, when the said Gs protein pathway dysfunction is a Gs alpha protein dysfunction, the test preferably consists in detecting one or more alterations related to the Gs alpha protein or the GsXL alpha protein, the said alterations being selected from the group consisting of:

mutations or functional polymorphisms in the human GNAS1 coding sequence,

mutations or functional polymorphisms in the human GNAS1 regulatory sequence,

variations in the methylation pattern of the human GNAS1 locus,

variations in the expression level of a human GNAS1 encoded protein,

different splicings of the human GNAS1 transcript,

mutations or functional polymorphisms in an alternative human GNAS1promoter, and

mutations in a modifying protein of the human GNAS1 locus. Methods for determining these mutations and variations are discussed above.

For instance, the GNAS1 coding sequence mutation may be a mutation within the triplet coding for Arg231 of Gs alpha protein, preferably a mutation resulting in a Arg231Cys mutation in the Gs alpha protein.

The GNAS1 coding sequence mutation may also be an insertion into the internal repeat region of the GsXL protein encoded by the human GNAS1 gene and/or one or more substitutions within said internal repeat region. It may also be a 36 bp insertion leading to a duplication of repeat 7 or repeat 8 structure of the internal repeat region of the GsXL protein encoded by the human GNAS1 gene and/or a substitution of Ala138 with Asp and/or a substitution of Pro161 with Arg in the GsXL protein.

According to this test or diagnostic method, when the said Gs protein pathway dysfunction is a Gs beta protein dysfunction, it may be identified by means of a genetic assay consisting in detecting one or more alterations in a Gs beta protein or a gene encoding said protein.

As a result of the above, this invention also provides a diagnostic kit for determining Gs protein pathway dysfunction, the said kit being selected from the group consisting of:

kits comprising compounds for the quantitative determination of Gs protein or fragments thereof; such as monoclonal and polyclonal antibodies against GNAS1 encoded proteins

kits comprising compounds for the quantitative determination of Gs mRNA; such as primers for performing RT-PCR

kits comprising compounds for the determination of Gs mRNA splice variants; such as one or more primer pairs for specifically amplifying DNA fragments only occurring in a said splice variant.

kits comprising compounds for the determination of Gs anti sense RNA; such as primer pairs for specifically amplifying anti sense RNA

kits comprising compounds for the detection of Gs mutations in the exons and/or introns and 5′ and 3′ regulatory elements; such as sequence specific primers which can discriminate wild type over mutated sequences, sequence primers, primers and probes for Southern hybridization, compounds for performing a protein trunaction.

kits comprising compounds for the detection of methylation within the human GNAS1 locus such as probes for Southern hybridization and methylation sensitive restriction enzymes.

The present invention also provides a method of treatment of a child with a condition of abnormal short stature in order to increase the growth rate of the said child, the said condition being characterized by a Gs protein pathway dysfunction identifiable by a functional or genetic assay, the said method comprising administration to the said child of an effective amount of human Growth Hormone, or a functional variant thereof. Preferably the said condition does not further include human Growth Hormone deficiency, as may be characterized by standard tests well known to those skilled in the art. Also preferably, the said condition of abnormal short stature should be characterized by a body weight and/or length which, at start of treatment, is more than two standard deviations below average of the relevant control population. The Gs protein pathway dysfunction pertinent to said method of treatment may be is a Gs alpha protein dysfunction or a Gs beta protein dysfunction.

An effective amount of the human Growth Hormone, or functional variant thereof, for the treatment of the invention is preferably an amount between 36 and 64 μg per kg bodyweight per day. Also preferably, administration is effected subcutaneously or intramuscularly or by percutaneous micro-injection, as is well known to those skilled in the art. According to conventional practice for this kind of treatment, the human Growth Hormone, or functional variant thereof, active ingredient may be formulated in the form of an injectable aqueous solution, for instance in admixture with standard amounts of adjuvants such as monosodium phosphate, disodium phosphate, amino-acetic acid and the like. As is also well known to those skilled in the art, when the relevant child is a girl, treatment should preferably be interrrupted in case of pregnancy.

The present invention is described in further details by reference to the following examples which are merely illustrative but not limiting the scope of the invention.

### EXAMPLE 1

### Treatment of a Girl Started at the Age of 47 Months

A girl was born by section after a 38 weeks gestation complicated by oligohydramnios. The parents are healthy and unrelated; father's height is 172 cm, mother's 163 cm. Birth weight was 2,240 grams (i.e. 2.4 standard deviations below average), birth length was 46 cm (i.e. 1.6 standard deviations below average), head circumference was 32 cm. The neonatal phase was characterized by generalized edema and feeding difficulties with oliguria. Early psychomotor development was normal.

This girl came to attention at the age of 17 months because of growth failure. At this age, length was 67.5 cm (i.e. 5.0 standard deviations below average), weight was 6.8 Kg (i.e. 5.6 standard deviations below average) and head circumference was 45 cm. Physical appearance was noteworthy for frontal bossing, blepharophimosis, depressed nasal bridge and small umbilicus; the acra were short with broad thumbs/halluces, impression of short metacarpals and bilateral clinodactyly of the fifth finger.

Peak serum Growth Hormone concentration was 10 ng/mL (a response excluding conventional Growth Hormone deficiency) during a standard glucagon test performed at the age of 18 months; cortisol responses were normal. Thyroïd stimulating hormone and prolactin presented so-called hypothalamic responses to thyroïd releasing hormone, their serum responses peaking respectively to 29.7 mU/I and 28 μg/L after 40 minutes. Serum IGF-I levels ranged from 22-43 ng/mL between 18-27 months. Magnetic Resonance Imaging of the pituitary gland visualized no abnormalities. Karyotype was 46 XX. Bone maturation was delayed for chronological age.

At the age of 27 months, the girl still had short stature and was diagnosed as having a Gₛ pathway disorder by documenting—through thrombocyte aggregation-inhibition testing—that there was a loss of G protein mediated signalling by three inducers (prostaglandin E1, iloprost and adenosine).

Molecular analysis of GNAS1, the gene encoding for the G protein alpha sub-unit, revealed a missense mutation in exon 9 (GAC to AAC in codon 231 resulting in a Arg to Cys mutation).

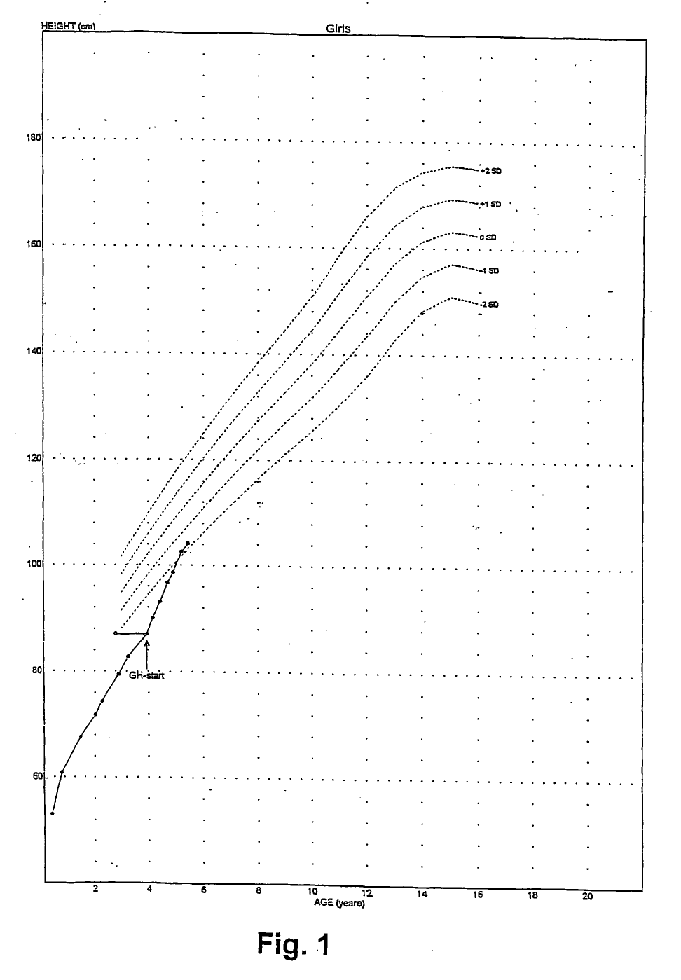

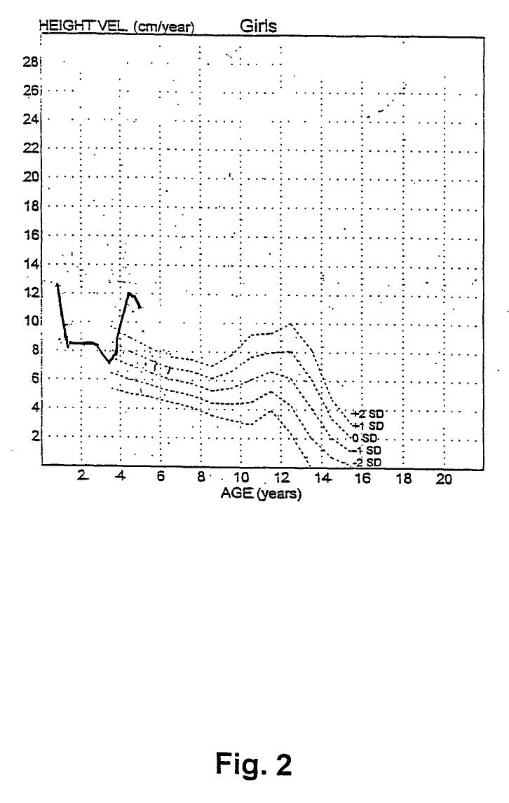

At the age of 47 months, Growth Hormone treatment was initiated at a dose of 50 μg per kg bodyweight per day in an attempt to normalize stature. This treatment has now proven successful, as evidenced by the attached growth charts (FIGS. 1 and 2): during the 18 months of Growth Hormone treatment, the child presented catch-up growth to within normal range.

### EXAMPLE 2

### Group Study

The aforementioned observation on the efficacy of Growth Hormone treatment in growth failure associated with G-protein pathway dysfunction has been confirmed by findings obtained over 12 months in a randomized, controlled study involving 12 pre-puberty children. Specific criteria required for inclusion into this group were:

1. A height (measured by means of a Harpenden stadiometer) being at least 2 standard deviations below average;

2. G-protein pathway dysfunction identified by the above-described platelet aggregation-inhibition test.

Children were randomized for one of two options: either stay untreated for one year, or receive treatment with recombinant human Growth Hormone (Somatotropin; 50 μg per kg bodyweight per day). The study protocol was approved by the Institutional Review Board of the University of Leuven. (Belgium). Written informed consent of at least one of the parents was obtained prior to study initiation.

At start of study, key average characteristics of the group were:

| | |
|---|---|
| Age (year) | 5.6 |
| Height Standard Deviation Score (SDS) | −2.8 |
| Growth Velocity (cm/year) | 6.2 |

After 12 months without or with Growth Hormone treatment, the main indicia were as indicated in the following table:

| | TABLE | |
|---|---|---|
| | Group | |
| | Untreated (n = 6) | GH-Treated (n = 6) |
| Change in Height (SDS) | 0.0 | 1.0 |
| Growth Velocity (cm/year) | 5.5 | 10.1 |

### EXAMPLE 3

### Treatment of a Boy Started at the Age of 10.3 Years

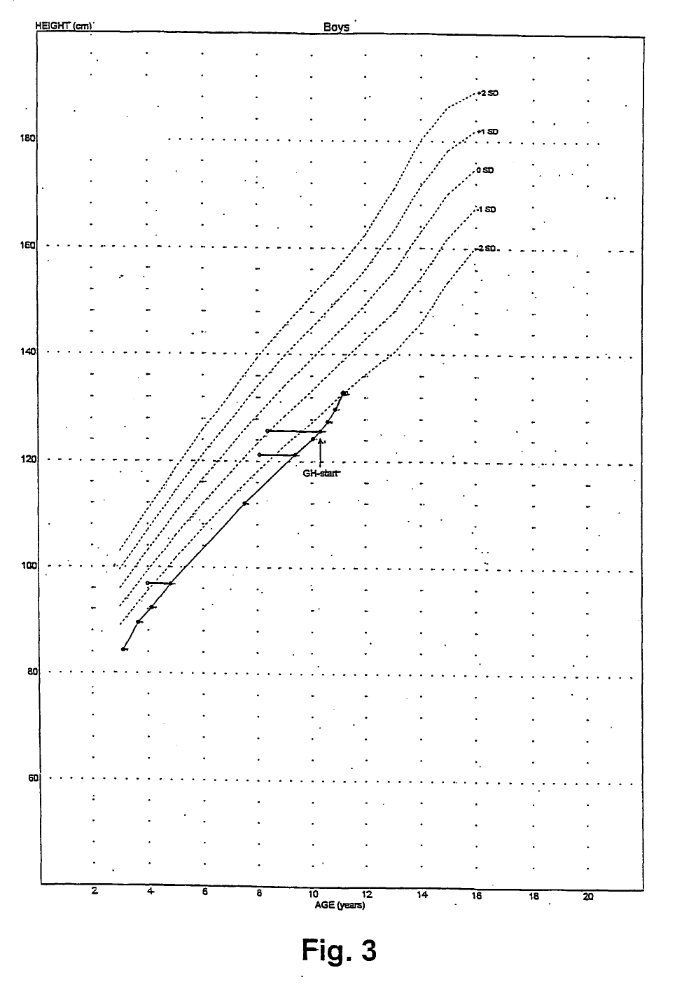

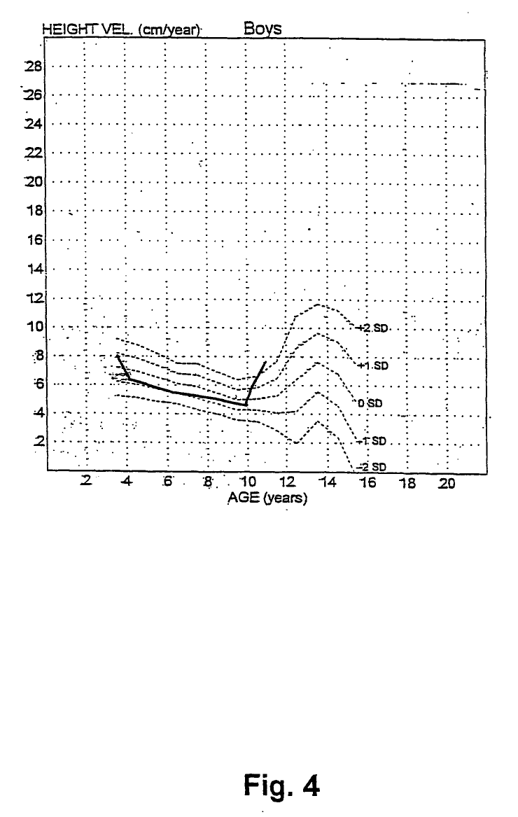

The treatment of example 2 was repeated with a boy-with platelet Gs-hyperfunction and enhanced cAMP generation upon stimulation of Gs-coupled receptors-who was found to have a known functional polymorphism in the imprinted XL-GNAS1 gene, consisting of a 36 bp insertion and of two bp substitutions flanking this insertion in the paternally inherited XL-GNAS1 exon 1. This boy was born after a 40 weeks gestation. The parents are healthy and unrelated; father's height is 187 cm, mother's 161 cm. Birth weight was 3,090 grams, birth length was 45 cm, head circumference was 35 cm. This boy came to attention at the age of 7.5 years because of growth failure. At this age length was 112 cm (i.e. 2.7 standard deviations below average). Treatment with recombinant human Growth Hormone at a dose of 50 μg per kg bodyweight per day was started at the age of 10.3 years. Results of the treatment over a period of nine months are shown in FIGS. 3 and 4: height growth rate increased to 7.8 cm/year, as compared to a growth rate of only 4.6 cm/year during the 12 months before treatment.

As a conclusion, the findings of the above examples evidence the fact that:

a fraction of children with hitherto unexplained short stature present a subtle down- or up-regulation of Gₛ-protein mediated signaling; and

the growth of children with such a variant of short stature is readily responsive to a treatment with an effective amount of exogenous Growth Hormone.

1. Use of human Growth Hormone, or a functional variant thereof, for the manufacture of a medicament for the treatment of a child with a condition of abnormal short stature in order to increase the growth rate of the said child, wherein the said condition is characterized by a Gs protein pathway dysfunction identifiable by a functional or genetic assay.

2. Use according to claim 1, wherein the said condition is further characterized by human Growth Hormone deficiency.

3. Use according to claim 1, wherein the said condition does not further include human Growth Hormone deficiency.

4. Use according to any of claims 1 to 3, wherein the said condition of abnormal short stature is characterized by a body weight and/or length at birth which is more than two standard deviations below average of the relevant control human population.

5. Use according to any of claims 1 to 4, wherein the said Gs protein pathway dysfunction is a Gs alpha protein dysfunction.

6. Use according to any of claims 1 to 4, wherein the said Gs protein pathway dysfunction is a Gs beta protein dysfunction.

7. Use according to any of claims 1 to 6, wherein the said functional assay comprises a thrombocyte aggregation-inhibition test indicating a change in G protein mediated signaling by at least one inducer.

8. Use according to claim 7, wherein the said functional assay comprises a thrombocyte aggregation-inhibition test indicating a loss in G protein mediated signaling by two inducers.

9. Use according to claim 7 or claim 8, wherein the said inducers are selected from the group consisting of prostaglandin E1, iloprost and adenosine.

10. Use according to any of claims 1 to 6, wherein the said functional assay comprises the steps of:
(f) providing a tissue or cell source from the said child with a condition of abnormal short stature,
(g) contacting the said tissue or cells obtained from the said cell source with one or more agonists or antagonists of Gs protein-coupled receptors,
(h) determining the response to all said agonists or antagonists of Gs protein-coupled receptors in the said cells or tissue,
(i) comparing the response obtained in step (c) with a reference response to all said agonists or antagonists of Gs protein-coupled receptors in the cells or tissue of a control human population, and
(j) determining whether the response obtained in step (c) is significantly different from the said reference response.

11. Use according to claim 10, wherein the said agonists or antagonists of Gs protein-coupled receptors are platelet-aggregation antagonists.

12. Use according to claim 11, wherein the said platelet-aggregation antagonists are selected from the group consisting of prostaglandin E1, adenosine and chemically stable prostacycline analogues (such as for instance iloprost, cicaprost, ataprost, beraprost, ciprostene, taprostene, naxaprostene and the like).

13. Use according to any of claims 1 to 6, wherein the said genetic assay consists in detecting one or more alterations in one or more Gs proteins or in one or more genes encoding the said Gs proteins or components of the said genes.

14. Use according to claim 13, wherein the said Gs protein pathway dysfunction is a Gs alpha protein dysfunction and wherein the said genetic assay consists in detecting one or more alterations related to the Gs alpha protein or the GsXL alpha protein, the said alterations being selected from the group consisting of:
mutations or functional polymorphisms in the human GNAS1 coding sequence,
mutations or functional polymorphisms in the human GNAS1 regulatory sequence,
variations in the methylation pattern of the human GNAS1 locus,
variations in the expression level of a human GNAS1 encoded protein,
different splicings of the human GNAS1 transcript,
mutations or functional polymorphisms in an alternative human GNAS1promoter, and
mutations in a modifying protein of the human GNAS1 locus.

15. Use according to claim 14, wherein the GNAS1 coding sequence mutation is a mutation within the triplet coding for Arg231 of Gs alpha protein.

16. Use according to claim 14 or claim 15, wherein the GNAS1 coding sequence mutation results in a Arg231Cys mutation in the Gs alpha protein.

17. Use according to claim 14, wherein the GNAS1 coding sequence mutation is an insertion into the internal repeat region of the GsXL protein encoded by the human GNAS1 gene and/or one or more substitutions within said internal repeat region.

18. Use according to claim 14 or claim 15, wherein the GNAS1 coding sequence mutation is a 36 bp insertion leading to a duplication of repeat 7 or repeat 8 structure of the internal repeat region of the GsXL protein encoded by the human GNAS1 gene and/or a substitution of Ala138 with Asp and/or a substitution of Pro161 with Arg in the GsXL protein.

19. Use according to claim 13, wherein the said Gs protein pathway dysfunction is a Gs beta protein dysfunction and wherein the said genetic assay consists in detecting one or more alterations in the Gs beta protein or a gene encoding said protein.

20. Use according to any of claims 1 to 19, wherein the human growth hormone, or a functional variant thereof, is used in the said treatment in an amount between 36 and 64 μg per kg bodyweight per day.

21. Use according to any of claims 1 to 20, wherein the said child with a condition of abnormal short stature is aged at least 2 years.

22. Use according to any of claims 1 to 21, wherein the said child with a condition of abnormal short stature is in a condition where epiphyseal growth plates are not fused.

23. A test method for use in diagnosing a disease or abnormal condition related to a Gs protein pathway dysfunction in a human being, the test being carried out on a tissue sample or a cell source from a human being, wherein the test comprises detecting one or more alterations in one or more Gs proteins or in one or more genes encoding the said Gs proteins or components of the said genes.

24. A diagnostic method for diagnosing a Gs protein pathway dysfunction or a disease or abnormal condition related thereto in a human being, comprising the step of performing onto the said human being, or a tissue sample or a cell source thereof, at least a genetic test consisting in detecting one or more alterations in one or more Gs proteins or in one or more genes encoding the said Gs proteins or components of the said genes.

25. A method according to claim 23 or claim 24, wherein the said abnormal condition is an abnormal short stature characterized by a body weight and/or length which is more than two standard deviations below average of the relevant control human population.

26. A method according to any of claims 23 to 25, wherein the said Gs protein pathway dysfunction is a Gs alpha protein dysfunction and wherein the test consists in detecting one or more alterations related to the Gs alpha protein or the GsXL alpha protein, the said alterations being selected from the group consisting of:
mutations or functional polymorphisms in the human GNAS1 coding sequence,
mutations or functional polymorphisms in the human GNAS1 regulatory sequence,
variations in the methylation pattern of the human GNAS1 locus,
variations in the expression level of a human GNAS1 encoded protein,
different splicings of the human GNAS1 transcript,
mutations or functional polymorphisms in an alternative human GNAS1promoter, and
mutations in a modifying protein of the human GNAS1 locus.

27. A method according to claim 26, wherein the GNAS1 coding sequence mutation is a mutation within the triplet coding for Arg231 of Gs alpha protein.

28. A method according to claim 26 or claim 27, wherein the GNAS1 coding sequence mutation results in a Arg231Cys mutation in the Gs alpha protein.

29. A method according to claim 26, wherein the GNAS1 coding sequence mutation is an insertion into the internal repeat region of the GsXL protein encoded by the human GNAS1 gene and/or one or more substitutions within said internal repeat region.

30. A method according to claim 26 or claim 27, wherein the GNAS1 coding sequence mutation is a 36 bp insertion leading to a duplication of repeat 7 or repeat 8 structure of the internal repeat region of the GsXL protein encoded by the human GNAS1 gene and/or a substitution of Ala138 with Asp and/or a substitution of Pro161 with Arg in the GsXL protein.

31. A method according to claim 26, wherein the said Gs protein pathway dysfunction is a Gs beta protein dysfunction and is further identifiable by a genetic assay consisting in detecting one or more alterations in a Gs beta protein or a gene encoding said protein.

32. A diagnostic kit for determining Gs protein pathway dysfunction, being selected from the group consisting of:
kits comprising compounds for the quantitative determination of Gs protein or fragments thereof;
kits comprising compounds for the quantitative determination of Gs mRNA;
kits comprising compounds for the determination of Gs mRNA splice variants;
kits comprising compounds for the determination of Gs anti sense RNA;
kits comprising compounds for the detection of Gs mutations in the exons and/or introns and 5′ and 3′ regulatory elements; and
kits comprising compounds for the detection of methylation within the human GNAS1 locus.

33. A method of treatment of a child with a condition of abnormal short stature in order to increase the growth rate of the said child, the said condition being characterized by a Gs protein pathway dysfunction identifiable by a functional or genetic assay, the said method comprising administration to the said child of an effective amount of human Growth Hormone, or a functional variant thereof.

34. A method of treatment according to claim 33, wherein the said condition does not further include human Growth Hormone deficiency.

35. A method of treatment according to claim 33 or claim 34, wherein the said condition of abnormal short stature is characterized by a body weight and/or length which, at start of treatment, is more than two standard deviations below average of the relevant control population.

36. A method of treatment according to any of claims 33 to 35, wherein the said Gs protein pathway dysfunction is a Gs alpha protein dysfunction.

37. A method of treatment according to any of claims 33 to 35, wherein the said Gs protein pathway dysfunction is a Gs beta protein dysfunction.

38. A method of treatment according to any of claims 33 to 37, wherein the effective amount is an amount between 36 and 64 μg per kg bodyweight per day.

39. A method of treatment according to any of claims 33 to 38, wherein administration is effected subcutaneously or intramuscularly or by percutaneous micro-injection.
